(19) United States
(12) Patent Application Publication
(10) Pub. No.: US 2012/0058164 A1
(43) Pub. Date: Mar. 8, 2012

(54) CLEANING OR COSMETIC COMPOSITIONS COMPRISING NATURAL ZEOLITES

(75) Inventors: Ray John Merchant, Napier (NZ); Paul Lap Shun Chan, North Shore Auckland (NZ); Philip John Ashenden, Auckland (NZ)

(73) Assignee: ZEOSOFT LIMITED, Auckland (NZ)

(21) Appl. No.: 13/141,684

(22) PCT Filed: Dec. 23, 2009
(86) PCT No.: PCT/NZ2009/000303

§ 371 Date: Nov. 14, 2011

(30) Foreign Application Priority Data
Dec. 23, 2008 (NZ) NZ573986

(51) Int. Cl. A61K 8/02; A61K 9/00; A61P 17/00; A61K 33/06; A61Q 19/00; A61K 8/26; A61K 9/14

(52) U.S. Cl. 424/401; 424/684; 424/400; 424/489

(57) ABSTRACT

The invention describes compositions which are useful as cleansers of the skin. The compositions described comprise zeolitic ingredients which are in general softer than known products. The natural zeolitic ingredients are inherently soft. The softness is due to a number of factors including but not limited to younger zeolitic rock types, softer constituent elements, more porous rock types and less dense rock types. The overall softness provided by the compositions according to the invention provides a cleaning product that is softer to the skin, especially the human skin.

### FIELD OF THE INVENTION

This invention relates to cleaning and cosmetic compositions. More particularly, but not exclusively, the present invention relates to soaps and the like products for use in cleaning and detoxifying skin, especially human skin.

### BACKGROUND OF THE INVENTION

The alkali-earth elements calcium and magnesium effectively prevent strong lathering and decrease the detergent power in soaps. The elimination or binding of these elements is important for detergents to perform adequately. Prior to the mid 1980s, laundry soap/detergent compositions included compounds to soften the water and bind these so called hard elements. These compounds are called detergent builders and, mostly because of cost, sodium tri-ammonium phosphate and related compounds were chosen.

However, the drawback of using phosphates became apparent in the 1980s when studies showed that phosphate overload was creating problems in effluent streams where prolific algal growth was occurring choking the effluent waterways. Zeolites emerged as alternatives. “Zeolite” is the collective name given to a group of over 50 natural and 200 artificial mineral species which have a wide range of industrial applications. Zeolites are crystalline, hydrated minerals which contain silica, aluminium and oxygen together with alkali and alkaline-earth metals such as sodium, potassium, magnesium, and calcium. Their structure is based on a three dimensional honeycomb framework of silicon and oxygen atoms forming tetrahedral patterns. The crystal framework is hollow. The framework contains pores and channels which carry negative charges. In its natural state these charges are balanced by metal cations such as calcium, magnesium, potassium and sodium ions. Around 50 zeolite species have been identified as naturally occurring but most industrially used zeolites are synthetic. Zeolites became the detergent building compounds of choice in the bulk of commercial products.

Zeolites have also been found to have other applications. In Patent application U.S. 2008/0004203 A1 it is proposed that zeolites can be added to cleaning products to assist in the process of wastewater detoxification and resisting bio fouling.

Aside from improved effluent characteristics in comparison with the use of phosphates, various additional benefits of adding zeolites to cleaning products are known. The internal channels permit the adsorption of toxins and malodors. The adsorbed compounds may be exchanged with the metal cations within the honeycomb structure and themselves become bound to the exposed negative charges. Thus in use in soaps and the like, zeolites can effectively “mop up” and lock in undesirable compounds, especially malodorous gases.

When used herein, “soap” means an alkali-fatty acid based cleansing product, or a like product in a similar form and/or intended for similar uses to an alkali-fatty acid soap, including but not limited to hand washes and hand cleaners containing surfactants, which may have some or all of its detergent properties due to materials or compounds other than alkali-fatty acid compounds, and which may be in the form of a bar, flowing liquid, paste or gel.

Patents such as U.S. Pat. Nos. 4,265,777 and 5,225,100 reveal formulations in solid, liquid, and particulate form of soaps with zeolites in conjunction with various surfactant types derived from long chain fatty acids. The zeolites (overwhelmingly synthetic and mostly Zeolite A or P) bind the calcium and magnesium and act as builders, water softeners, and free flow agents in powders. U.S. Pat. No. 4,230,590 to Colgate-Palmolive refers to zeolites in soaps which are said to also act as anti-static agents, and fabric softeners; U.S. Pat. No. 5,133,899 to Mizusawa Industrial Chemicals further refers to the use of zeolites as formulation stabilizers. U.S. Pat. No. 5,211,870 to Proctor and Gamble refers to zeolite addition for deodorizing purposes in bar soaps. U.S. Pat. No. 6,893,632 and related patents describe the addition of zeolites to various soaps and detergents for the capture of human body odours during hunting. They discuss the addition of zeolite to laundry water, to mouthwashes, body powders, and personal deodorants, specifically for use in hunting.

Because of the need for a constant composition and whiteness in cleaning compositions, synthetic zeolites have tended to be preferred over the cheaper but variable natural zeolites. In the literature on natural zeolites, there is often no clear cut reason as to why one zeolite is used over another. However, there has been a noticeable preference to use clinoptilolite where the field is medical or cosmetic, whereas mordenite is common in instances where odour control, or sewage treatments are issues (Eyde and Holmes in Industrial Minerals and Rocks 2006). Where absorption of fluids or modification of a zeolite is made, clinoptilolite is the zeolite of choice.

Much of the work on natural zeolites has been on varieties found physically close to, and with ready access to appropriate markets. For example, the clinoptilolite deposits are common world-wide and are mined in the United States are where they are widely used and well understood. These, and the vast proportion of natural zeolites around the world, formed in metamorphic conditions where entrained groundwaters, heated by the natural earth processes associated with burial of rocks at depth, interacted with certain components of the host rock (notably volcanic glass), and recrystallised it into particular zeolites depending on the temperature and pressure at that depth. This material is loosely called zeolite; commonly in such deposits the “zeolite”, more correctly described as zeolitic rock, is in fact a mixture of zeolite, quartz, clays, silica species and other residual minerals in the original lithology.

The zeolitic rock hitherto used in existing cleaning compositions is typically old. For example, 4-30 million years old in the case of zeolite ores from the United States, Indonesia, Europe, Japan or more than 300 million years old in the case of NSW Australian deposits. In mineral deposit of this age, all minerals present reflect conditions in which temperatures and pressures have been elevated but usually static for long periods. Old natural zeolitic rock tends to have a lower porosity due to long periods of burial and compression; this reduces its capacity to take up fluid molecules including water. This renders some of the U.S. zeolitic rocks unsuitable for direct use in absorbents such as kitty litter. Further, silica which is found in association with zeolites in these ancient deposits tends to be substantially, if not totally, converted to cristobalite or quartz, which are very hard and abrasive. As a consequence, such zeolitic rock or ore is hard and of simple mineralogy with few accessory mineral phases, and is relatively unsuitable for applications involving use against skin, especially human skin, such as soaps and cosmetics, in comparison to synthetic zeolites. On the other hand, synthetic zeolites tend to be relatively homogenous “zeolite which lacks other additional desirable mineral characteristics, such as the ability to supply active silica to the skin. Also, synthetic zeolites, which tend to consist of smooth round and equi-sized granules, are ineffective for exfoliation of dead skin and removal of ground-in oils.

### OBJECT OF THE INVENTION

It is an object of the present invention to provide cleaning compositions incorporating zeolites which overcome at least some of the abovementioned problems associated with older or harder or denser or less porous or otherwise unsuitable zeolitic material, or at least to provide the public with a useful choice.

### SUMMARY OF THE INVENTION

The invention provides a cleaning or cosmetic composition comprising one or more natural zeolites in which the zeolitic rock particles are soft.

The softness can be defined by high porosity and low specific gravity of the source rock.

The invention also provides a cleaning or cosmetic composition comprising one or more natural zeolites, wherein, due to the inherent softness factors or the source rock, particles from the zeolitic rock will present as if the Mohs hardness overall is the equivalent of less than 5.

More preferably the equivalent softness is less than Mohs 4 and most preferably the softness is less than Mohs 3.5.

The invention also provides a soft cleaning or cosmetic composition comprising one or more natural zeolites.

Particles are obtained from the zeolitic rock by methods known to those skilled in the art. Such methods include but are not limited to one or more of crushing, grinding, milling, screening, sieving, filtering and such methods. Any one or more of them are herein referred to generically as “milled”.

The softness can be defined by the age of the zeolitic rock.

The invention also provides a cleaning or cosmetic composition and/or a method of manufacturing a cleaning or cosmetic composition, comprising one or more natural zeolites obtained from a deposit less than 30 million years old.

Preferably the one or more natural zeolites is/are obtained from a deposit less than 10 million years old and most preferably from a deposit less than 1 million years old.

According to a further aspect of the invention there is provided a cleaning or cosmetic composition and/or a method of manufacturing a cleaning or cosmetic composition, comprising one or more natural zeolites obtained from a deposit less than 100,000 years old.

More preferably the one or more natural zeolites is/are obtained from a deposit less than 50,000 years old

The softness can be defined by the nature of the zeolite formation.

According to a further aspect of the invention there is provided a cleaning or cosmetic composition and/or a method of manufacturing a cleaning or cosmetic composition, comprising one or more natural zeolites formed by geothermal fluids that are related to volcanic processes.

The softness can be defined by the porosity of the source rock.

According to a further aspect of the invention there is provided a cleaning or cosmetic composition and/or a method of manufacturing a cleaning or cosmetic composition, comprising a milled zeolitic rock with a rock porosity of above 20%.

According to a further aspect of the invention there is provided a cleaning or cosmetic composition and/or a method of manufacturing a cleaning or cosmetic composition, comprising a milled zeolitic rock with a rock porosity of above 25%.

According to a further aspect of the invention there is provided a cleaning or cosmetic composition and/or a method of manufacturing a cleaning or cosmetic composition, comprising a milled zeolitic rock with a rock porosity of above 35%.

According to a further aspect of the invention there is provided a cleaning or cosmetic composition and/or a method of manufacturing a cleaning or cosmetic composition, comprising a milled zeolitic rock with a rock porosity of above 45%. The porosity may be greater than 60%.

The softness can be defined by the specific gravity of the zeolitic rock.

According to a further aspect of the invention there is provided a cleaning or cosmetic composition and/or a method of manufacturing a cleaning or cosmetic composition, comprising a milled zeolitic rock with an original uncrushed specific gravity of less than 2.4 g/ccm, preferably less than 2.2 g/ccm, more preferably less than 2.0 g/ccm and most preferably less than 1.5 g/ccm.

The softness of the zeolitic rock can be defined by the hardness of the mineral constituents.

The softness of the zeolitic rock can be defined by the low crystal sizes of these mineral constituents including but not restricted to one or more of the zeolite(s) in the rock.

Preferably the crystal sizes are substantially less than 50 microns, more preferably less than 25 microns and more preferably less than 20 microns.

According to a further aspect of the invention there is provided a cleaning or cosmetic composition wherein said zeolitic rock is soft as indicated by a combination of high porosity and low specific gravity.

There may be two or more natural zeolites. Preferably the two or more natural species of zeolite are coeval zeolites.

According to a further aspect of the invention there is provided a soft cleaning or cosmetic composition and/or a method of manufacturing a cleaning or cosmetic composition, comprising two or more zeolitic minerals.

More preferably the zeolitic minerals are clinoptilolite and mordenite.

Preferably the zeolites may comprise about 0 to about 95% by weight clinoptilolite and about 0 to about 95% by weight mordenite.

Preferably the ratio by weight of clinoptilolite: mordenite may be at most about 1:1 and more illustratively about 1:2.

The softness can be defined by the amount and type of non-quartz metastable forms of silicon dioxide (silicas) present in the zeolitic rock.

According to a further aspect of the invention there is provided a cleaning or cosmetic composition and/or a method of manufacturing a soft cleaning or cosmetic composition, comprising one or more natural zeolites and further comprising one or more non-quartz metastable forms of silicon dioxide (silicas).

Preferably said non-quartz metastable forms of silicon dioxide comprise one or more mineral species selected from amorphous silica (the soft form Opal A) and Opal C-T. As used herein Opal C-T refers to a form of silicon dioxide with elements of the structure of the minerals Cristobalite and Tridymite. Opal C is cristobalite.

Preferably the composition comprises a soft zeolitic rock of which less than about 80% by weight is non-quartz metastable forms of silicon dioxide by weight, more preferably about 10 to about 50%, most preferably about 5 to about 25%.

The non-quartz metastable forms of silicon dioxide may include a form for which Mohs hardness is <6.5, preferably <5, more preferably<4 and most referably <3.5.

Opal A may dominate the—non-zeolite assemblage.

Preferably the silicas are in intimate association with said one or more zeolites. Preferably the silicas are derived from the same source rock material as the one or more zeolites.

The softness can be indicated by the amount and type of quartz.

Preferably the soft composition comprises a zeolitic rock which is substantially free of, or comprises less than, about 20 percent by weight, quartz including both hydrothermal and original magmatic origin.

More preferably the zeolitic rock comprises less than 15% quartz, more preferably less than 10% quartz more preferably less than 5% quartz and most preferably is substantially free of quartz.

Preferably the quartz grains, if present are of primary magmatic origin and are substantially rounded and embayed, hence of low abrasivity.

The softness can be defined by the amount and type of mineral clays present in the zeolitic rock.

According to a further aspect of the invention there is provided a soft cleaning or cosmetic composition and/or a method of manufacturing a soft cleaning or cosmetic composition, comprising one or more natural zeolites and further comprising one or more mineral clays such as, but not restricted to the smectite group of clays, more preferably calcium smectite clay.

The clay may be derived from the same source rock material as the one or more zeolites.

According to at least one form of the invention, said composition comprises a zeolitic rock of which about 0 to about 40% by weight is said clay. Preferably the zeolitic rock comprises about 3 to about 10% by weight of clay.

The clay may be derived from the same source rock material as one or more of the zeolites.

The invention also provides soft cleaning and/or cosmetic composition comprising a milled zeolitic rock in which about 0 to about 30%, preferably about 0-15%, by weight of the particles of said rock are between about 300 and about 500 micron in size.

Preferably about 30 to about 100%, preferably about 50 to about 95%, by weight of the particles of said rock are smaller than about 150 micron.

More preferably less than about 10%, preferably about 3-5%, by weight, of the particles of said rock are larger than about 500 micron.

Most preferably about 1 to about 95%, preferably about 1 to about 40%, by weight, of the particles of said rock are between about 1 and about 10 micron in size.

The invention provides a soft cleaning or cosmetic composition comprising one or more natural zeolites wherein the hydrothermal assemblages of the zeolitic source rock are obtained from volcanic-related geothermal processes in the Quaternary volcanic arc of the central North Island of New Zealand and more preferably from Ngakuru deposits in New Zealand.

The cleaning or cosmetic composition is preferably a soap. The soap may be a bar soap, flowing liquid soap, gel soap or paste soap.

The invention also provides a method of preparation of the above cleaning or cosmetic compositions.

The invention will now be described, by way of example only with reference to specific embodiments which are intended to be illustrative of the invention and not limiting.

### DETAILED DESCRIPTION OF THE INVENTION

It has surprisingly been found that cleaning compositions which comprise zeolites from deposits which are under 1 million years old, illustratively less than about 100,000, most illustratively less than about 50,000 years old, termed herein, “young” zeolites, have certain advantageous characteristics.

It has further unexpectedly been found that selection of source zeolitic rock in accordance with certain parameters as described herein leads to an end product of superior performance in the cleaning and cosmetic fields. Further it has been found that by selective processing via grading of the material, further advantages are accrued.

It has been found that “young” zeolites, or more correctly young zeolitic rocks tend to offer a more open structure and are highly reactive. This can enhance the ability of the compositions made from them to adsorb both toxins and odour causing compounds.

It has been found that these young zeolitic rocks can have higher porosity and this translates into a soft zeolitic rock with especial properties suited to the skin cleaning and skin care field.

It has further been found further that these young zeolitic rocks have lower specific gravities and this also translates into a soft zeolitic rock with especial properties suited to the skin cleaning and skin care field.

It has been learned that young, open, porous, low specific gravity natural zeolitic material when incorporated into cleaning compositions are softer and less abrasive. It has been learned further that their heterogeneous shapes make them better absorbers of oils and fluids and can also offer significant exfoliation via gentle scouring of superficial skin layers.

It has also been found that zeolite from young deposits, particularly young deposits formed from fluids derived by volcanic rather than metamorphic processes may also have a zeolite mineralogy and a chemistry that is of variable composition. Some young zeolite rock is of relatively simple mineralogy containing mordenite zeolite and silica species opal C-T.

However, it has unexpectedly been discovered that improved performing soaps can be made using material which contains a mixture of zeolites for example clinoptilolite and mordenite.

Different species of zeolite will have different pore sizes. We have learned that the use of two or more zeolitic species increases the range of molecules, such as toxins and malodorous species which will enter the honeycomb structure of the zeolites and potentially be adsorbed. Further, using zeolites of more than one species leads to heterogeneous crystal shapes which was found to improve cleaning efficacy.

Further, the variable mineralogy of the source rock may desirably offer additional mineral and chemical content which improves the performance of the cleaning compositions, for example through interaction with ionic (charged) species which may be present on skin.

Rather than quartz, silica in the zeolitic rock used according to at least one illustrative embodiment of the invention is illustratively dominantly composed of non-quartz species, such as amorphous silica (Opal A), Opal C-T and/or Opal C. Possibly as a result of the pulsation of thermal episodes in the past, the zeolite minerals in certain young deposits are coeval and intimately intergrown with silica for example with silica species Opal A and C-T. The presence of these minerals in the zeolitic rock, particularly Opal-A has been found to confer certain advantageous properties on cleaning compositions, especially soaps, made using these zeolites. Compared to quartz, these non-quartz silicas are less abrasive against skin, and, as is known by prior art, may act as skin conditioners and softeners.

An important advantage of this mineralogy is that of silica reactivity, especially that of silica species with surfactants. These silica species are metastable and highly reactive forms of silicon dioxide. In the presence of alkaline surfactants this metastable silica is activated and becomes ionically available to the mix; this enables polymeric silica bearing species to form and these may have benefits for skin conditions.

The zeolitic rock may also include mineral clays such as but not restricted to the smectite group of clays, which together with the zeolite species may have significant cationic exchange capacity i.e. the ability to exchange cations from the mineral surface for other cationic species, which may be undesirable cationic species, and to hold them bound in the composition, thus having a cleaning or purifying effect.

It has further been found in the present invention that zeolitic material in which the ingredients of zeolites and/or silicas and/or clays are coeval, i.e. are all the same geological age and naturally and intimately intergrown, performs better than if these ingredients are sourced separately and simply mixed at the time of product manufacture. However, separate sourcing is also possible and is included within the scope of at least one form of the invention.

One source of natural zeolite for use according to the invention is a tuffaceous lacustrine siltstone of the Ngakuru Formation in the central areas of the Ngakuru Graben, Rotorua, New Zealand. This rock was deposited in ancient lakes between about 20,000 and 250,000 years ago and these lakes were filled with volcanic debris from local sources. Contemporary and later stage volcanic-related geothermal processes set up thermal spas in the district. The water circulating in these springs below the surface caused modification of the original volcanic rock, in particular its volcanic glass (in the tuffs) to form zeolites, silica species and locally clays. The components of the altered tuff are: zeolites, (about 40-85%) non quartz silica species (about 20-50%), mineral clays (about 5-25%), alkali feldspar (0-10%) and plagioclase feldspar and/or quartz (about 0-10% in total). The last two minerals are relicts of the original rock.

The zeolite assemblages and parageneses in these New Zealand deposits, more specifically the Ngakuru deposits, are in some respects different from those commonly found elsewhere. They are very young, and unchanged by later burial. They are volcanic, hosted in young tuffs, and very porous and non-compacted. The fluid causing the mineral deposition was volcanic-geothermal in origin. In contrast, the common zeolite deposits globally are older (commonly Miocene age or older), are the products of fluid action from waters of immediate local derivation at these great depths (metamorphic and diagenetic), and are hard and usually of low porosity.

This deposit has zeolite crystals that are very small, mostly below 10-15 microns. This we found advantageous as it allowed for the softness of the material to be preserved via the silica species and clays. It further allowed for larger particles of the rock to be functional as toxin and malodour adsorbers even in larger particles as the tiny zeolite crystals are part of the larger particles containing softer material. It is known to those skilled in the art that zeolites are highly effective at smaller particle sizes as this exposes large surface areas.

The Ngakuru deposits contain significant mordenite which is known as one of the softer of the zeolite group; this contributes to the overall softness of the material used from those deposits.

The presently illustrative zeolitic rock for use according to the present invention is from a younger less deeply buried deposit than that previously known or used. Judging by the presence of surface features still being recognizable viz sulphur fumaroles, sinter terrace material and eruption craters, the thermal activity here probably at least younger than 100,000 years and more likely less than 50,000 years old; in fact there are active thermal spas less than 10 km away.

It has further been unexpectedly discovered that improved cleaning performance comes from use of a carefully graded zeolitic material. It is possible to achieve specific attributes in the end product composition by using specific grain sizing of the zeolitic material.

Also it has been found that in certain instances a small percentage of larger grain is desirable for exfoliation. Material of larger graded size provides sufficient grains of suitable size to scour out built up grime and oils dried into grooves in the hand. By removing said accumulations, far better cleaning and deodorizing is possible. We have found however that having the bulk of the material at lower sizing bolsters odour removal and adsorption. The finer fraction can absorb and trap oils and adsorb any malodour. As will be apparent to those skilled in the art, a finer grading is required to allow flowing or liquid soaps to be delivered through nozzles without blocking. Further with body soaps and cosmetic cleaners, too high a particle size can result in abrasion of sensitive skin. Illustratively, a flowing soap product may suitably be made in which all or substantially all of the zeolitic material is smaller than about 300 microns.

We have found that material in the 300-500 micron range is effective for exfoliation of dead skin and most importantly can clear built up oils and greases which accumulate in the troughs between the ridges of the hand. We found that in at least one illustrative form of the invention, it is particularly effective to have about 15-25% by weight of the milled zeolite in the composition in the range 300-500 microns but most preferably about 15-20% in that range.

Milled zeolitic particles smaller than about 150 microns are thought to do the bulk of the adsorption of toxins, including heavy metals, and malodorous compounds as they have a particlarly large open surface area of charges.

We found that it is particularly effective to have about 40-95% by weight of the milled zeolite in the composition, preferably about 50-95%, smaller than about 150 micron.

Furthermore, a finely graded material may be used in compositions for more cosmetic purposes. It is known in scientific literature that the larger the surface area of the zeolite the higher the capacity to adsorb compounds such as malodours. It also follows that the finer the grind of the zeolite the better the product will be at soaking up fine oil droplets or residual films of the breakdown products of the combustion of petroleum and tobacco; such compounds are not uncommon on the skin of city dwellers. By using a fine zeolite (less than 30 micron, and preferably less than 10 micron) a soap can be fashioned specifically intended as a cosmetic cleaning soap (see examples 3, 9 and 10), not intended to exfoliate, but rather meant to ad- and absorb oils, toxins and malodours. Micronization below about 10 microns may also make amorphous silica and/or smectite clay, if present, more available in the composition; and the zeolites more active.

Thus in some embodiments, a very small cut in the 1-10 micron range, suitable for nourishing the skin post exfoliation, can be used. Illustratively, the milled zeolitic rock in the composition can have about 1 to 30% by weight of particles in the <1 to 10 micron range. Illustratively, the milled zeolitic rock in the composition can have about 1 to 10% by weight of particles in the 1 to 10 micron range. Alternatively, for example, if use in mild soaps or sensitive skin applications is required, then larger sizes may be more rigorously excluded. Illustratively, in some cosmetic applications more than about 80%, or in some cases approximately 100%, of the zeolitic material may suitably be smaller than about 10 microns.

Milled particles larger than about 500 micron can assist with “heavy” scrubbing of skin. We found that it is particularly effective to have less than about 10% of the milled zeolite in the composition, preferably about 5%, larger than about 500 micron.

The source rock for Example 1 has about 5% by weight of particles in the 500-700 micron range giving coarser scouring, about 30% in the 300-500 micron range giving good cleaning and also exfoliation, about 52% are smaller than about 150 micron and particles in this range are believed to interact with the skin giving deodorizing and toxin removal properties, with about 13% of the fine sizing of 1-10 micron, suitable for nourishing the skin post exfoliation. After further milling, screening and sorting as required, zeolitic material can be selected and compiled for use in compositions according to the desired weightings of particular size ranges.

It should be noted, as will be apparent to those skilled in the art that the amount of zeolitic material within the composition will vary depending on the desired end product form. For example, in a mild product such as a shower body wash, the zeolitic material may be less than 10% of the total composition. In a mid-range composition, around 10-35% may be appropriate. Higher percentages, for example 35-50% or above 50% are also envisaged for very strong products. These examples are illustrative.

### EXAMPLES

Example formulations according to the invention will now be described. It will be appreciated the invention incorporates many other possible formulations and these examples are not intended to be limiting.

It will also be appreciated that although particular sources of zeolite suitable for use according to the invention have been described, the invention is not limited to use of zeolite from these particular deposits. Other deposits may potentially prove to be suitable for use according to the invention and use of relatively young zeolite as herein defined from those sources is envisaged and incorporated in the present invention.

### Example 1

### Bar Soap

In this example, soap is fashioned from natural palm soap noodles and other soap ingredients mixed with 18% by overall product weight of particles derived from young zeolitic rock. The natural fine-grained zeolites and related coeval hydrothermal minerals in intimate association in this same-source material are <50,000 years old and formed from volcanic-related geothermal processes in the Quaternary volcanic arc of the central North Island of New Zealand.

The material has a composition of about 28% tabular clinoptilolite zeolite, 42% acicular mordenite zeolite, 20% non-quartz metastable forms of silicon dioxide being Opal A (soft amorphous silica) Opal C-T and minor Opal C, with Opal A>Opal C-T>Opal C, 5% mineral clays in this case smectite clay, and up to 5% embayed relic quartz.

The source rock has a specific gravity of <1./5 g/ccm and a porosity or >45%. This is an example of material with both low density (specific gravity) and high porosity. Less than 5% of the zeolitic rock is of materials with Mohs hardness >4. It contains soft non-quartz metastable silicon dioxide material with Mohs 2.5-3. The zeolite crystals are 90% <25 micron in greatest dimension. Because of various softness factors (including but not limited to density, porosity and constituent materials) particles from this zeolitic rock will present as if the Mohs hardness overall is the equivalent of <2.5. The material can be declared to be soft and is well suited for this application.

In this example the particle size distribution chosen of the milled screened and cleaned zeolitic material is 7% >500 microns, 18% 300-500 microns, 24% 150-300 microns, 18% 50-150 microns, 18% 10-50 microns, 15% <10 microns.

Natural colorants and perfumes are added to improve the delivery of the soap to the customer. The soap is made via known methods of mixing and extrusion into molds, as will be known by those versed in the art of soap manufacture.

### Example 2

### Variant Bar Soap

In this example also, soap is fashioned from natural palm soap noodles and other soap ingredients mixed with 18% by overall product weight of particles derived from young zeolitic rock. The natural fine-grained zeolites and related coeval hydrothermal minerals in intimate association in this same-source material are <50,000 years old and formed from volcanic-related geothermal processes.

The material has a composition of about 30% tabular clinoptilolite zeolite, 30% acicular mordenite zeolite, 30% non-quartz metastable forms of silicon dioxide being Opal A (soft amorphous silica) and Opal C-T with Opal A predominating, 5% mineral clays in this case smectite clay, and up to 5% rounded embayed relic quartz. Compared to Example 1, this material has a different ratio of mordenite and clinoptilolite, and elevated content of soft silica.

The source rock has a specific gravity of <1.5 g/ccm and a porosity of >50%. This is another example of material with both low density (specific gravity) and high porosity. Less than 10% of the zeolitic rock is of materials with Mohs hardness >4. It contains soft non-quartz metastable silicon dioxide material with Mohs 2.5-3. The zeolite crystals are 90% <25 micron in greatest dimension.

Because of various softness factors (including but not limited to density, porosity and constituent materials) particles from this zeolitic rock will present as if the Mohs hardness overall is the equivalent of <2-2.5. The material can be declared to be soft and is well suited for this application.

In this example the particle size distribution chosen of the milled screened and cleaned zeolitic material is 3% >500 microns, 10% 300-500 microns, 32% 150-300 microns, 10% 50-150 microns, 30% 10-50 microns, 15% <10 microns.

Natural colorants and perfumes are added to improve the delivery of the soap to the customer. The soap is made via known methods of mixing and extrusion into molds, as will be known by those versed in the art of soap manufacture.

### Example 3

### Cosmetic Bar Soap

This example is a bar soap suited for cosmetic or sensitive skin applications. In this example soap is fashioned from natural palm soap noodles and other soap ingredients mixed with 20% by overall product weight of particles derived from soft young geothermally formed zeolitic rock.

The material has a composition of 18% tabular clinoptilolite zeolite, 46% acicular mordenite zeolite, 23% non-quartz metastable forms of silicon dioxide being Opal A (soft amorphous silica) and Opal C-T with Opal A predominating, 5% mineral clays in this case smectite clay, and up to 8% in total of very fine grained potash feldspar and rounded embayed relic quartz. It is an instance of a material with a low clinoptilolite to mordenite ratio. The source rock has a specific gravity of <1.7 g/ccm and a porosity of >40%. This is another example of material with both low density (specific gravity) and high porosity.

Less than 10% of the zeolitic rock is of materials with Mohs hardness >4. It contains >20% soft non-quartz metastable silicon dioxide material with Mohs 2.5-3. The zeolite and potassium feldspar crystals are 90% <25 micron in greatest dimension. In this example the particle size distribution chosen of the milled screened and cleaned zeolitic material is 97% <10 microns, 3% 10-50 microns. Because of various softness factors particles from this zeolitic rock will present as if the Mohs hardness overall is the equivalent of 2.5-3. The material can be declared to be soft and is well suited for this application.

The product is otherwise manufactured substantially as in Examples 1 or 2.

### Example 4

### Flowing Liquid Handwash

In this example a handwash product is produced from suitable surfactants and other handwash ingredients mixed with 10% by overall product weight of particles derived from soft young geothermally formed zeolitic rock.

The coevally formed material derived from the zeolitic rock that is used in this example composition is 55-60% acicular mordenite zeolite, 25% non-quartz metastable forms of silicon dioxide being Opal A (soft amorphous silica) and Opal C-T with low Mohs, Opal A predominating, up to 10% of very fine grained (90% <15 microns) very fine grained alkali feldspar, <10% in total of rounded embayed relic quartz and plagioclase, and some mineral clay. This rock has a specific gravity of <1.0 g/ccm and a porosity of >45%, and is another example of both high porosity and low density. The fine-grained zeolite and alkali feldspar crystals are 90% <25 micron in greatest dimension.

In this example the particle size distribution chosen of the milled screened and cleaned zeolitic material is 5% 150-300 microns, 70% 50-150 microns, 5% 10-50 microns, 20% <10 microns.

The composition contains a base capable of supporting the milled particles as will be understood by those skilled in the known art. The zeolitic material is pre-treated prior to inclusion in the batch by washing in a manner well known to those skilled in this field.

### Example 5

### Variant Flowing Liquid Handwash

In this example a handwash product is produced from suitable surfactants and other handwash ingredients mixed with 12% by overall product weight of particles derived from zeolitic rock aged 10--25 million years (Miocene age). The material derived from the zeolitic rock that is used in this example has 65% clinoptilolite 15% Mordenite, 16% Opal A and C-T, and 5% quartz. This rock has a specific gravity of <2 g/ccm and a porosity of 30-35%. It contains non-quartz metastable silicon dioxide material with Mohs <5. Because of various softness factors, particles from this zeolitic rock will present as if the Mohs hardness overall is the equivalent of
<3.5-4. In this example the particle size distribution chosen of the milled screened and cleaned zeolitic material is 5% 150-300 microns, 45% 50-150 microns, 10% 10-50 microns, 40% <10 microns.

The product is otherwise manufactured substantially as in Example 4.

### Example 6

### Gel (Low Viscosity Paste) Hand Cleaner

In this example a hand cleaner composition in a gel form (higher viscosity than a flowing liquid handwash, lower viscosity than a paste) is produced from suitable surfactants and other handwash ingredients mixed with 15% by overall product weight of particles derived from zeolitic rock. The age of zeolitization is about 4-5 million years.

The material derived from the zeolitic rock that is used in this example has 70-75% clinoptilolite zeolite (no mordenite), with the remainder being Opal C and rounded embayed quartz. The quartz comprises 5-10%. The specific gravity is <2.2 g/ccm and porosity <55%. The rock has 10% minerals with Mohs above 4. It contains non-quartz metastable silicon dioxide material with Mohs <4. The equivalent Mohs hardness of the composite material is estimated to be below 4.

In this example the particle size distribution chosen of the milled screened and cleaned zeolitic material is 5% less than 300 microns, 50% 150-300microns, 15% 50-150 microns, 10% 10-50 microns, 20% less than 10microns.

The composition contains a base capable of supporting the milled particles as will be understood by those skilled in the known art. The zeolitic material is pre-treated prior to inclusion in the batch by washing in a manner well known to those skilled in this field.

### Example 7

### Paste Hand Cleaner

This product was developed for use in a range or applications where the cleaning task calls for a more “heavy duty” capability than is usually needed for general household hand cleaning. In this example a hand cleaner composition is produced from suitable surfactants and other handwash ingredients mixed with 35% by overall product weight of particles derived from a Miocene aged zeolitic rock (in this case aged between 25 and 30 million years).

The material derived from the zeolitic rock that is used in this example has 75-80% clinoptilolite zeolite (no mordenite), with the remainder being Opal C microcrystalline quartz, relict quartz and about 5% clays. The specific gravity is about 2.3 g/ccm and porosity is low, at 24-30%. Approximately 15-20% of the material has Mohs >4, making this zeolitic material less soft than that used in some of the other examples.

The fine-grained zeolite crystals are 90% <50 micron in greatest dimension. In this example the particle size distribution chosen of the milled screened and cleaned zeolitic material is 7% >500 microns, 18% 300-500 microns, 24% 150-300 microns, 18% 50-150 microns, 18% 10-50 microns, 15% <10 microns.

The base of the paste has adequate suspension capability for the viscosity and would be easily concocted by those versed in the art.

### Example 8

### Variant Paste Hand Cleaner

This product was, as for example 7, developed for use in a range of niche applications where the cleaning task calls for a more “heavy duty” capability than is usually needed for general household hand cleaning. In this example a hand cleaner composition in a paste form similar to Example 7 is produced from suitable surfactants and other handwash ingredients mixed with 35% by overall product weight of particles derived from soft natural fine-grained zeolites and related coeval hydrothermal minerals in intimate association. The rock is <50,000 years old and formed from volcanic-related geothermal processes.

The material has a composition of about 5% tabular clinoptilolite zeolite, 35% acicular mordenite zeolite, 35% non-quartz metastable forms of silicon dioxide being Opal A, C-T and C, 10% mineral clay, 10% fine-grained feldspar and 5% rounded embayed relic quartz. The source rock has a specific gravity of <1.2 g/ccm and a porosity of >60%. This example is of one with higher clay content, low specific gravity and high porosity.

In this example the particle size distribution chosen of the milled screened and cleaned zeolitic material is 6% >500 microns, 20% 300-500 microns, 24% 150-300 microns, 17% 50-150 microns, 18% 10-50 microns, 15% <10 microns.

The composition contains a base capable of supporting the milled particles as will be understood by those skilled in the known art. The zeolitic material is pre-treated prior to inclusion in the batch by washing in a manner well known to those skilled in this field.

### Example 9

### Variant Cosmetic Bar Soap

This example is a bar soap suited for cosmetic or sensitive skin applications. In this example also, soap is fashioned from natural palm soap noodles and other soap ingredients mixed with 20% by overall product weight of finely sized particles derived from young zeolitic rock containing abundant clays and other soft materials. The natural fine-grained zeolites and related coeval hydrothermal minerals in intimate association in this same-source material are <120,000 years old and formed from volcanic-related geothermal processes.

The material has a composition of about 30% tabular clinoptilolite zeolite, 20% softer acicular mordenite zeolite, 27% Opal A (soft amorphous silica) and some Opal C-T with Opal A predominating, 20% mineral clays in this case smectite clay, and minor amounts of Opal C and embayed relic quartz.

The geothermally altered source volcanogenic rock has a specific gravity of <1.6 g/ccm and a porosity of 55%. This is another example of material with both low density (specific gravity) and high porosity.

Less than 5% of the zeolitic rock is of materials with Mohs hardness >4. It contains soft non-quartz metastable silicon dioxide material with a Mohs hardness of 2.5-3. The zeolite crystals are 90% <20 micron in greatest dimension; they have Mohs hardnesses of about 3 and the clays have Mohs of about 1.5-2. Because of various softness factors (including but not limited to density, porosity and constituent materials) and the fact that most of the constituents have hardnesses of 3 or below, particles from this zeolitic rock will present as if the Mohs hardness overall is the equivalent of <2.5. The material can be declared to be soft and is well suited for this application.

In this example the particle size distribution chosen of the milled screened and cleaned zeolitic material is 0.25% 50-300 microns, 5% 10-50 microns, 94.75% <10 microns, producing an effective yet gentle soap.

The product is an example of a soap with grain sizings adjusted for minor exfoliation properties; otherwise it is manufactured substantially as in Example 1.

### Example 10

### Variant Cosmetic Bar Soap

This example is another bar soap suited for cosmetic or sensitive skin applications. In this example also, soap is fashioned from natural palm soap noodles and other soap ingredients mixed with 22% by overall product weight of finely sized particles derived from soft young zeolitic rock. The natural fine-grained zeolites and related coeval hydrothermal minerals in intimate association in this same-source material are <50,000 years old and formed from volcanic-related geothermal processes.

The material has a composition of about 30-40% tabular clinoptilolite zeolite, 10-15% softer acicular mordenite zeolite, 50-55% Opal A (soft amorphous silica) and Opal C-T, 5% Opal C, 5% mineral clays in this case smectite clay, and 8% total of fine grained feldspar and rounded embayed relict quartz. It is an instance of a soft material with high concentrations of soft Opals.

The source rock has a specific gravity of <1.5 g/ccm and a porosity of >55%. This is another example of material with both low density (specific gravity) and high porosity. In this example the particle size distribution chosen of the milled screened and cleaned zeolitic material is 97% <10 microns, 3% 10-50 microns. The product is otherwise manufactured substantially as in Example 1.

### Example 11

### Variant Gel (Low Viscosity Paste) Hand Cleaner

In this example a hand cleaner composition in a gel form similar to Example 6 is produced from suitable surfactants and other handwash ingredients mixed with 22% by overall product weight of particles derived from soft natural fine-grained zeolites and related coeval hydrothermal minerals in intimate association. The rock is <50,000 years old and formed from volcanic-related geothermal processes.

The material has a composition of about 30% tabular clinoptilolite zeolite, 40% acicular mordenite zeolite, 30% non-quartz metastable forms of silicon dioxide being Opal A, C-T and C, 10% mineral clay, 3-5% embayed relic quartz and minor fine grained feldspar. The source rock has a specific gravity of <1.75 g/ccm and a porosity of >45%.

Less than 5% of the zeolitic rock is of materials with Mohs hardness >4. It contains soft non-quartz metastable silicon dioxide material with Mohs <2.5-3. The zeolite crystals are 90% <25 micron in greatest dimension.

Because of various softness factors (including but not limited to density, porosity and constituent materials) particles from this zeolitic rock will present as if the Mohs hardness overall is the equivalent of <2.5-3. The material can be declared to be soft and is well suited for this application. In this example the particle size distribution chosen of the milled screened and cleaned zeolitic material is 3% >500 microns, 10% 300-500 microns, 30% 150-300 microns, 12% 50-150 microns, 30% 10-50 microns, 15% <10 microns.

The composition contains a base capable of supporting the milled particles as will be understood by those skilled in the known art. The zeolitic material is pre-treated prior to inclusion in the batch by washing in a manner well known to those skilled in this field. Where in the foregoing description reference has been made to integers or components having known equivalents then such equivalents are herein incorporated as if individually set forth.

Although this invention has been described by a way of example of possible embodiments, it is to be appreciated that improvements and/or modifications may be made thereto without departing from the scope of the present invention.

### INDUSTRIAL APPLICABILITY

The invention relates to a cleaning or cosmetic composition which will find a use for washing in many situations. The compositions described are soft to the human skin and hence of interest to the public as washing products which are less abrasive and kinder to the skin than some known products.

1.-69. (canceled)

70. A cleaning or cleansing composition including cosmetic compositions whose functions include cleaning or cleansing and scrubbing the skin, the composition being adapted to be applied to the skin, the composition comprising milled zeolitic rock particles with one or more natural zeolites wherein said zeolitic rock is soft.

71. A composition according to claim 70 which comprises at least 3% by weight of zeolitic rock.

72. A composition according to claim 70 in which the softness is defined by a high porosity and low specific gravity.

73. A composition according to claim 70 wherein, due to the inherent softness factors, particles from the zeolitic rock present as if the Mohs hardness overall is the equivalent of less than 5, or less than Mohs 4 or less than Mohs 3.5.

74. A composition according to claim 70 in which the natural zeolite is obtained from a deposit less than 30 million years old, or less than 10 million years old, or less than one million years old, or less than 100,000 years old, or less than 50,000 years old.

75. A composition according to claim 70 wherein the one or more natural zeolites is/are formed by geothermal fluids that are related to volcanic processes, or is/are obtained from volcanic-related geothermal processes in the Quaternary volcanic arc of the central North Island of New Zealand, optionally wherein the zeolitic rock is obtained from Ngakuru deposits in New Zealand.

76. A cleaning or cosmetic composition according to claim 70 wherein said zeolitic rock has an original uncrushed porosity of greater than 20%, or greater than 30%, or greater than 45%, or greater than 60%, optionally wherein said zeolitic rock has an original uncrushed specific gravity of less than 2.4 g/ccm, or less than 2.2 g/ccm, or less than 2.0g/ccm, or less than 1.5 g/ccm.

77. A composition according to claim 70 wherein said zeolitic rock is soft as defined by the hardness of the mineral constituents, optionally wherein said zeolitic rock is soft as defined by the low crystal sizes of one or more of the mineral constituents including zeolite(s) in the zeolitic rock, optionally wherein the zeolite crystal sizes are predominantly of less than 50 microns, or less than 25 microns, or less than 20 microns.

78. A composition according to claim 70 comprising two or more natural species of zeolite, optionally wherein said two or more natural species of zeolite are coeval zeolites, or include clinoptilolite and mordenite, optionally wherein said zeolites comprise about 0 to about 95% by weight clinoptilolite and about 0 to about 95% by weight mordenite, optionally in which the ratio by weight of clinoptilolite: mordenite is about 1:1 and more illustratively about 1:2.

79. A composition according to claim 70 further comprising one or more non-quartz metastable forms of silicon dioxide, optionally wherein said non-quartz metastable forms of silicon dioxide comprise one or more mineral species selected from amorphous silica (Opal A), Opal C (Cristobalite) and Opal C-T, optionally wherein said composition comprises a zeolitic rock of which less than about 80% by weight is one or more of said non-quartz metastable forms of silicon dioxide, optionally in which the zeolitic rock comprises one or more non quartz metastable forms of silicon dioxide with Opal A being present in a greater amount than any other non-quartz metastable forms of silicon dioxide, optionally in which less than 60% is one of said non-quartz metastable forms of silicon dioxide, optionally in which 5-50% is non quartz metastable forms of silicon dioxide, optionally in which 10-25% is non quartz metastable forms of silicon dioxide.

80. A composition according to claim 70 wherein said non-quartz metastable forms of silicon dioxide are in intimate association with said one or more zeolites, optionally wherein said non-quartz metastable forms of silicon dioxide are derived from the same source rock material as said one or more zeolites.

81. A composition according to claim 70 wherein said non-quartz metastable forms of silicon dioxide include a form for which Mohs hardness is <6.5, preferably <5, more preferably <4, and most preferably <3.5

82. A composition according to claim 70 wherein the zeolitic rock comprises substantially less than about 20% quartz, optionally less that 15% quartz, optionally less than 10% quartz, optionally less than 5% quartz, optionally which is substantially free of quartz by weight, of either hydrothermal or magmatic origin, optionally in which the quartz grains of magmatic origin are rounded and embayed.

83. A composition according to claim 70 which comprises one or more natural zeolites and mineral clay such as but not restricted to smectite group clays in particular calcium smectite clay, optionally wherein about 0 to about 40% by weight is said clay, optionally in which about 3-10% is clay, optionally wherein said clay is derived from the same source rock material as said one or more of the zeolites.

84. A composition according to claim 70 comprising a milled zeolitic rock, in which about 0 to about 30% by weight of the particles of said zeolitic rock are between about 300 and about 500 microns in size, optionally in which about 10-25% of the particles are between about 300 and 500 microns in size.

85. A composition according to claim 70 comprising a milled zeolitic rock, in which about 0 to about 60% by weight of the particles of said zeolitic rock are between about 150 and about 300 micron in size

86. A composition according to claim 70 comprising a milled zeolitic rock, in which about 30 to about 100% by weight of the particles of said zeolitic rock are smaller than about 150 micron, optionally in which 50 to 95% of particles are smaller than about 150 microns, optionally in which about 30-100% by weight of the particles of said zeolitic rock are smaller than about 50 microns, optionally in which about 10-100% by weight of the particles of said zeolitic rock are smaller than about 10 microns, optionally in which about 1 to about 95%, preferably about 1 to about 40%, by weight, of the particles of said rock are between about 1 and about 10 microns in size.

87. A composition according to claim 70 comprising a milled zeolitic rock in which less than about 10% by weight of the particles of said zeolitic rock are larger than about 500 microns.

88. A composition according to claim 70 in a solid form, optionally in the form of a soap such as a bar soap or a powder soap.

89. A composition according to claim 70 in a non-solid form, optionally in the form of a soap such as a gel, or a flowing liquid soap, or a paste soap.
